(19) United States
(12) Patent Application Publication
(10) Pub. No.: US 2018/0023110 A1
(43) Pub. Date: Jan. 25, 2018

(54) METHOD FOR PREPARING FUNCTIONAL POLYPEPTIDE THROUGH MULTIMODE ULTRASONIC ENHANCING ENZYMOLYSIS

(71) Applicant: JIANGSU UNIVERSITY, Jiangsu (CN)

(72) Inventors: Haile MA, Jiangsu (CN); Xiaofeng REN, Jiangsu (CN); Cunshan ZHOU, Jiangsu (CN); Zhenbin WANG, Jiangsu (CN); Ronghai HE, Jiangsu (CN); Wenjuan QU, Jiangsu (CN); Yanyan ZHANG, Jiangsu (CN); Shuyun MAO, Jiangsu (CN)

(21) Appl. No.: 15/547,458

(22) PCT Filed: Jan. 21, 2016
(86) PCT No.: PCT/CN2016/071590

§ 371 Date: Jul. 28, 2017

(30) Foreign Application Priority Data
Jan. 30, 2015 (CN) 201510048637.2
Jan. 30, 2015 (CN) 201510052239.8
Mar. 27, 2015 (CN) 201510138570.1
Mar. 27, 2015 (CN) 201510138613.6

(51) Int. Cl. C12P 21/06; B01J 19/10

(52) U.S. Cl. CPC C12P 21/06; B01J 19/10

(57) ABSTRACT

The invention is about methods for preparing functional polypeptide by multi-mode ultrasound enhancement on enzymolysis, and it relates to the field of functional peptide production technologies. The objective of the invention is to improve the degree of hydrolysis of protein, increase the anti-hypertensive activity of protein hydrolysate, shorten the enzymolysis time and reduce the energy consumption. To achieve this, some methods are developed, such as the method for preparing the anti-hypertensive peptide from wheat gluten by sequential ultrasound enhanced enzymatic hydrolysis, preparation of anti-hypertensive peptide from corn gluten by ultrasonic reverberation field, and the technology for preparing functional polypeptide by countercurrent mode dual frequency ultrasound pretreatment of prion protein. The invention of solid-liquid scanning ultrasonic pretreatment of corn gluten meal suspension leads to an improved efficiency of enzymatic hydrolysis. The invention of multi-mode ultrasonic pretreatment of wheat gluten suspension also improves the enzymatic hydrolysis efficiency. Compared with the traditional enzymatic hydrolysis method, the effect of multi-mode ultrasonic pretreatment on the ACE inhibitory rate of enzymatic hydrolysate is increased by 16.4%˜25.4%, and compared with single frequency ultrasound, the ACE inhibitory rate of enzymatic hydrolysate is increased by 6.0%˜13.7%.

### THIS PCT REQUIRES THE FOLLOWING PATENT PRIORITY

201510048637.2 Method for preparing antihypertensive peptides from wheat gluten based on sequential ultrasonic enhanced enzymolysis.

201510052239.8 Method for preparing antihypertensive peptides from corn gluten based on ultrasonic reverberation field.

201510138613.6 Method for preparing ACE inhibitory peptides from wheat gluten by multi-mode ultrasonic wave strengthening enzymolysis.

201510138570.1 Method for treating gluten protein by countercurrent dual ultrasonic frequencies.

### TECHNICAL FIELD

The invention is a technology that relates to the field of functional peptide production technologies, especially the pretreatment of wheat gluten and corn protein material by multi-mode ultrasound, and then the method for preparing antihypertensive peptides by enzyme.

### BACKGROUND TECHNOLOGY

There is about 90 million tons of protein byproducts produced in China's agricultural products processing industry every year. Due to the high degree of degeneration, low solubility and poor nutritional quality, the protein by products cannot be fully utilized and this leads to serious waste of protein resources.

According to statistics, China's annual output of protein byproducts include 9 million 390 thousand tons of rapeseed meal; about one million tons of corn gluten meal and about 22 million 520 thousand tons of soybean meal. Enzymolysis of low protein into active peptide is an effective way to solve the problem of serious waste of protein resources.

Wheat gluten as a by-product of wheat starch processing contains the protein content of up to 75-80%, balanced amino acid composition, is rich in nutrition, cheap and high quality vegetable protein polymer. Wheat gluten in China is about 600,000 tons per year, at present, it is mainly used in the food and feed industry, such as flour quality improver, feed binder, nutrition additive and so on. According to new research, prion protein is a potential source of plant protein for preparing bioactive peptides, its enzymatic hydrolysate can markedly inhibit blood pressure by inhibiting the activity of angiotensin converting enzyme (ACE). However, due to the high content of hydrophobic amino acids (mainly Pro, Gln, Leu, Ala), the solubility of the protein is poor, which made it difficult for the protein to combine with the enzyme, thus, the efficiency of enzymatic hydrolysis is not high.

The production of Chinese corn ranks second in the world, with an annual output of about 25% of the world. Corn gluten meal is rich in protein, and contains 15 kinds of inorganic salts and unique yellow pigment and flavour. However, because of its lack of lysine, tryptophan and other essential amino acids, it is mainly used in the preparation of crude feed, the production of nutritional food additives, and direct treatment of emissions; and this is a great waste of resources. In recent years, there are many studies on protein hydrolysis (peptides). There are a lot of reports about the preparation of bioactive peptides from corn protein by protease hydrolysis. At present, a large number of ACE inhibitory peptides have been found in the hydrolysates of zein, often referred to as antihypertensive peptides, the results showed that these ACE inhibitory peptides showed good antihypertensive effect on spontaneously hypertensive patients, but there was no hypotensive effect in normotensive subjects. Corn protein contains a large number of hydrophobic groups, the solubility is poor, in the process of enzymatic hydrolysis, it is not easy to digest, the hydrolysis rate is slow, the efficiency of enzymatic hydrolysis and the conversion rate of product is low, and the product quality is unstable.

Ultrasound is a kind of high frequency (more than 20 kHz) longitudinal wave, small wavelength and strong penetration ability. The interaction between the ultrasonic wave and the medium produces cavitation effect. The cavitation effect can produce high temperature, high pressure, simultaneous shock wave in a small area, therefore, it is possible to destroy the spatial structure of the protein, so that a large number of amino acids are exposed, and the cleavage site is exposed, which is conducive to the later enzymatic hydrolysis, thereby improving the hydrolysis degree of protein byproducts. According to Mao Shuyun et al. (2013), the swept ultrasonic pretreatment of zein was performed before the alkaline protease was hydrolyzed, showed that the degree of hydrolysis of corn gluten increased from 12.5% to 45.6% compared to the conventional enzymolysis; Du Shuangkui et al. (2013). studied the pretreatment of chickpea with ultrasonic cleaning machine, adding alkaline protease enzymatic hydrolysis, the hydrolysis of chickpea increased from 20.03% to 25.04%; Ma Haile working group studied the probe-type ultrasonic pretreatment of oat protein. The results showed that the hydrolysis degree of oat protein in the treatment group was 15.8% higher than that in the control group.

Ultrasonic pretreatment of protein substrates can improve the enzymatic hydrolysis of wheat gluten, and improve product activity by changing the two level structure of protein, the size of protein molecules, and the degree of binding between molecules. However, ultrasonic processing time and working mode directly affect the change of protein structure. This change is closely related to the improvement on enzymatic properties and biological activities.

The study has shown that the combination of dual frequency or multi frequency ultrasound has obvious enhancement effect on the yield of sonochemical reaction, the combined effects of dual frequency or multi frequency ultrasound radiation were significantly greater than the sum of the individual radiation effects of each frequency. For example, a combination of 28 kHz and a 1.7 MHz dual frequency ultrasound radiation, when the ultrasonic intensity of 1.7 MHz was greater than 3 W/cm², the coupling effect was about 1.5 times that of the single effect; when the 1.7 MHz is changed to 1.06 MHz or 0.87 MHz, the coupling effect was about 2 times that of the single effect; a bunch of low frequency ultrasound is added on the basis of the dual frequency orthogonal radiation, constituting triband orthogonal irradiation, which can greatly improve sonochemical yield. The results of the three co-operation were about 1.4-1.8 times the sum of the three radiation yields. A study on the effect of different ultrasound frequencies on silver carp myofibrillar protein structure by “modern food science and technology” (Chinese, 2014, 30(3):23-26), Using silver carp as raw material, using single and dual ultrasound frequency respectively and triple treatment of silver carp myofibrillar protein, the effects of different ultrasonic frequency treatments on myofibrillar hydrophobic surface protein and surface hydrosulfuryl content were studied. Compared with the sample without ultrasound, after the triple frequency ultrasonic treatment for 10 min and single frequency ultrasonic treatment for 15 min, the surface hydrophobicity of protein increased by 65.34% and 56.88%, respectively, and the sulfhydryl content of protein surface also increased by 13.32% and 11.19%, respectively. Dual frequency ultrasonic, surface interaction between single and triple frequency ultrasonic treatments has effect on the hydrophobic surface protein thiol. This shows that the triple ultrasonography in protein modification has some advantages.

Most of the existing researches on multi frequency ultrasound are based on synchronous multi frequency mode, using the superposition effect of multi frequency ultrasonic peak to improve the effect of ultrasonic treatment. This effect occurs in multiple frequency ultrasonic generators work that simultaneously. This results in complex synthetic spectrum with the extension of treatment time, the peak addition and cancellation in different degrees.

Invention Content:

The purpose of the invention is to overcome the deficiency of the previous conventional methods, and provides a method for preparing functional polypeptide by multi-mode ultrasonic intensified enzymolysis, aimed to improve the degree of protein hydrolysis, improve the activity of protein hydrolysate, reduce the time of enzymatic hydrolysis, and reduce the energy consumption. The aim also includes the development of antihypertensive peptides with good efficacy, safety and affordable price.

The purpose of the invention is realized by the following technical means:

Specific Technical Scheme:

Plan One:

Method for preparing functional polypeptide by multi-mode ultrasonic wave strengthening enzymolysis method, and method for preparing blood pressure reducing peptide of protein of wheat gluten by sequential ultrasonic intensification, the following steps are carried out: (1) the gluten is formulated as a suspension with a mass concentration of 3%, and pretreated by the order of triple frequency ultrasound; (2) under the conditions of pH 9.0, temperature of 50° C., substrate concentration of 3%, alkaline protease enzyme dosage 4560 U/g, enzymatic hydrolysis for 30 min, use of 0.2 mol/L HCl to adjust pH to 7.0, enzyme inactivation in boiling water bath for 10 minutes, after cooling, centrifuge at 10000 g for 10 min, and then collecting the supernatant fraction, so far, the extract of protein antihypertensive peptide was obtained.

The order of triple frequency ultrasonic is 20 kHz, 28 kHz, 35 kHz, triple frequency ultrasonic time is 20 s, 20 s, 20 s, and the ultrasonic power per unit volume is 16.67 W/L, pretreatment time 10-16 min.

Plan Two:

The invention designs three interfaces of solid-liquid, liquid-liquid and gas-liquid, respectively to form ultrasonic reverberation field with different shapes, the solid-liquid interface is formed with a non working ultrasonic plate and water body, the liquid-liquid interface is formed with vegetable oil and water body and the gas-liquid interface is formed by air and water.

Method for preparing functional polypeptide by multi-mode ultrasonic wave strengthening enzymolysis, and method for preparing corn protein antihypertensive peptide by means of scanning frequency reverberation field ultrasonic strengthening enzymolysis, the following steps are carried out: (1) suspension with mass concentration of 4.5%, which is made from corn protein, and pretreated by the reverberation field ultrasonic; (2) under the conditions of pH 9.0, temperature of 50° C., substrate concentration of 4.5%, alkaline protease enzyme dosage of 5616 U/g, enzymatic hydrolysis for 60 minutes, use of 0.2 mol/L HCl to adjust pH to 7.0, enzyme inactivation in boiling water bath for 10 minutes, after cooling, centrifuge at 10000 g for 10 minutes, and then collecting the supernatant fraction, so far, the extract of protein antihypertensive peptide can be obtained.

Sweep frequency is 22±2 kHz, 28±2 kHz, 33±2 kHz, 40±2 kHz or 68±2 kHz.

Sweep frequency form reverberation field with different reflection media through the three interfaces of solid-liquid, liquid-liquid and gas-liquid, the solid-liquid interface is formed by metal plates and water bodies, the liquid-liquid interface is formed by vegetable oil and water, the gas-liquid interface is formed by air and water.

The ultrasonic reverberation field is preferably a solid-liquid reflecting medium, and the frequency of the swept ultrasonic reverberation field is: (40±2) kHz.

Ultrasonic pretreatment conditions include: the working time of pulsed ultrasound of 10 s and intermittent time of 3 s, ultrasonic sweep period of 500 ms, unit volume ultrasonic power of 120 W/L, initial temperature of 18 C, and pretreatment time of 30 minutes.

Plan Three:

In order to improve the anti-oxidative activity of the hydrolysates of wheat gluten protein and shorten the time of enzymatic hydrolysis, the protein was pretreated by countercurrent multi-mode ultrasonic equipment. The invention is realized by the following technical means:

Method for preparing functional polypeptide by multi-mode ultrasonic intensified enzymolysis, and method for preparing blood pressure reducing peptide of wheat gluten protein by counter current dual frequency ultrasound, the following steps are carried out: (1) Making wheat gluten protein into a suspension with mass concentration of 3% with dilute alkali solution with 0.003 mol/L, and pretreated by counter current dual frequency ultrasound; (2) under the conditions of pH 9.0, temperature of 50° C., alkaline protease (Alcalase) enzyme dosage of 4560 U/g, substrate concentration of 10 g/L, enzymatic hydrolysis for 30 minutes, use of 0.2 mol/L HCl to adjust pH to 7.0, enzyme inactivation in boiling water bath for 10 min, after cooling, centrifuge at 10000 g for 10 min, and then collecting the supernatant fraction, so far, the extract of protein antihypertensive peptide can be obtained.

The mode of operation of the ultrasonic is the order of dual frequency and synchronous dual frequency, frequency combination is 20-28, 20-35, 20-40 and 20-50 kHz, the frequency of the sequential operation is 20-35 kHz, and the working time is 5 s and pulsed time is 5 s, the frequency of the synchronous operation is 20-28 kHz, the pulse time is 10 s and the intermittent time is 5 s.

Ultrasonic pretreatment conditions include: the ultrasonic power per unit volume is 200 W/L, the initial temperature is 30° C., and the pretreatment time is 15 min.

The Advantages of the Invention are:

This invention adopts the ultrasonic pretreatment of the raw materials to study the ACE inhibitory activity of hydrolysates. After three frequency ultrasonic pretreatment, its effect on enzymatic hydrolysis is improved, the activity of enzymatic hydrolysate is improved, and compared with the original ultrasonic treatment, the energy consumption is also reduced. The ACE inhibitory activity of enzymatic hydrolysates increases from 13% to 20%. Energy consumption reduces by 3-53.75 times.

The invention adopts the method of solid-liquid scanning ultrasonic reverberation field to pretreat the suspension of corn protein powder. The ACE inhibitory activity of enzymatic hydrolysate after ultrasonic treatment was studied, The ACE inhibitory activity of enzymatic hydrolysates and DH increased by 44.9% and 37.6%, respectively. Compared with other types of ultrasonic pretreatment, the activity of the hydrolysates was improved. It was found that the phenomenon of emulsification easily occurred in the ultrasonic reverberation field formed at the liquid-liquid interface, which has certain value for the equipment design and industrial production. But, this kind of interface cannot be formed in the test and actual production (without ultrasound pretreatment).

This invention adopts the countercurrent multi-mode ultrasonic equipment to control the expression of prion protein. The results showed that the enzymatic hydrolysis efficiency was improved and the activity of the enzymatic hydrolysate was improved after the sequential double frequency ultrasonic pretreatment. Compared with the traditional enzymatic hydrolysis method, the ACE inhibitory activity of enzymatic hydrolysates was increased from 16.4% to 25.4%, compared with counter current single frequency ultrasounds, the ACE inhibitory activity of enzymatic hydrolysates was increased from 6% to 13.7%.

Following through the figures and examples, the technical scheme of the invention is further described in detail.

### DESCRIPTION OF FIGURES

FIG. 1 is the diagram of equipment of sequential tri band ultrasonic pretreatment of protein gluten used in the invention;

FIG. 2 is the ultrasonic reaction liquid-liquid transmission medium;

FIG. 3 is the ultrasonic reaction gas-liquid transmission medium;

FIG. 4 is the ultrasonic reaction solid-liquid transmission medium;

FIG. 5 is the diagram of equipment of countercurrent multi-mode dual frequency ultrasound pretreatment of prion protein used in the invention.

### SPECIFIC IMPLEMENTATION METHODS

In the invention, the degree of hydrolysis (DH) was used to measure the reaction efficiency of the whole reaction, and the degree of hydrolysis (DH) was determined by pH-stat method. Excessive angiotensin converting enzyme (ACE) in human body is one of the important reasons for the high blood pressure. Therefore, the invention is to evaluate the antihypertensive activity of ACE by inhibiting the enzymatic hydrolysate. The method of determination of ACE inhibitory activity of enzymatic hydrolysate by “The use of ultrasound for enzymatic preparation of ACE-inhibitory peptides from wheat germ protein, Jia et al, Food Chemistry. 119 (2010) 336-342” Take Hippuryl-His-Leu, HHL as a substrate for ACE catalysis, studying the change of absorbance, which is measured by HPLC method.

Plan One:

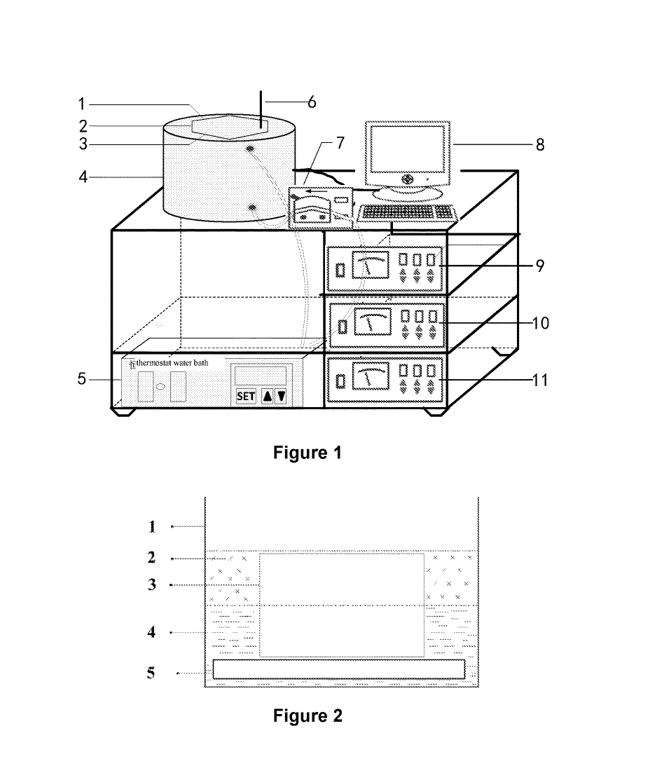

FIG. 1 is the diagram of the equipment of sequential tri band ultrasonic pretreatment gluten used in the invention, which was independently developed by Jiangsu University. Ultrasonic generators 9, 10 and 11 can emit ultrasonic waves of 20, 28 and 35 kHz, respectively, and the power of a single ultrasonic generator is 100 W. Ultrasonic vibration plate is vertically placed on the inner wall of ultrasonic pool 4, the ultrasonic vibration plates 1, 2, and 3 are respectively controlled by the ultrasonic generator 9,10 and the 11; After the computer controller 8 sets the ultrasonic parameters, control ultrasonic generator 9, 10, 11, issued in accordance with the requirements of ultrasound; 5 is the constant temperature water bath for the equipment of the invention, Through the thermometer 6, real-time monitoring of temperature, and according to the need to adjust the temperature of the media; The liquid material that needs to be processed is pumped into the ultrasonic pool 4 through the peristaltic pump 7 for ultrasonic treatment.

Control Group 1:

(1) Preparation of 2% (W/W) of the prion protein suspension of 500 mL volume, put into the 30° C. constant temperature water bath pot with rotor stirring 30 min, addition of 4560 U/g (E/S) alkaline protease was used for enzymatic hydrolysis, Enzymatic hydrolysis process was maintained at pH 9, temperature of 50° C., enzymolysis of 30 min, then boiling of enzyme in water bath for 10 min, 10000 g centrifugation for 10 min, supernatant collected, and was diluted 2 times to measure ACE inhibitory activity. After measurement, the inhibition rate of ACE of gluten hydrolysate was 64.957%.

(2) Preparation of 3% (W/W) of the prion protein suspension of 500 mL volume, put into the 20° C. constant temperature water bath pot with rotor stirring 30 min, addition of 4560 U/g (E/S) alkaline protease was used for enzymatic hydrolysis, enzymatic hydrolysis process was maintained at pH 9, temperature of 50° C., enzymolysis of 30 min, then boiling of enzyme in water bath for 10 min, 10000 g centrifugation for 10 min, supernatant collected, and was diluted 3 times to measure ACE inhibitory activity. After measurement, the inhibition rate of ACE of gluten hydrolysate was 66.012%.

(3) Preparation of 3% (W/W) of the prion protein suspension of 500 mL volume, was put into the 20° C. constant temperature water bath pot with rotor stirring for 50 min, addition of 4560 U/g (E/S) alkaline protease was used for enzymatic hydrolysis, Enzymatic hydrolysis process was maintained at pH 9, temperature of 50° C., enzymolysis time of 30 min, then boiling of enzyme in water bath for 10 min, 10000 g centrifugation for 10 min, collected supernatant, that is diluted 3 times to measure ACE inhibitory activity. After measurement, the inhibition rate of ACE of gluten hydrolysate was 64.761%.

(4) Preparation of 2% (W/W) of the prion protein suspension of 500 mL volume, single frequency probe type ultrasonic was applied to treat the wheat gluten protein suspension, the initial temperature was 30° C., the power was 896 W/L, the pulse time is 4 s, the interval time is 3 s, and the total ultrasonic time is 30 min. After ultrasound, addition of 4560 U/g (E/S) alkaline protease was used for enzymatic hydrolysis, enzymatic hydrolysis process was maintained at pH 9, temperature of 50° C., enzymolysis time of 30 min, then boiling of enzyme in water bath for 10 min, 10000 g centrifugation for 10 min, supernatant was collected and was diluted 2 times to measure ACE inhibitory activity. After measurement, the inhibition rate of ACE of gluten hydrolysate was 70.770%.

(5) Preparation of 2% (W/W) of the prion protein suspension of 500 mL volume, under the condition of mechanical agitation, the three divergent frequency ultrasonic of 20 kHz, 28 kHz and 35 kHz, was applied and the synchronous ultrasonic time was 10 s, the interval time was 5 s, the ultrasonic power was 50 W/L, the initial temperature was 30° C. to treat it for 30 min; enzymatic hydrolysis was carried out by adding alkaline protease of 4560 U/g, enzymatic hydrolysis process was maintained at pH 9, temperature of 50° C., enzymolysis time of 30 min, then boiling of enzyme in water bath for 10 min, 10000 g centrifugation for 10 min, supernatant was collected, and was diluted 2 times to measure ACE inhibitory activity. After measurement, the inhibition rate of ACE of gluten hydrolysate was 70.07%.

### Implementation Example 1

Preparation of 2% (W/W) of the prion protein suspension of 6 L volume, under the condition of mechanical agitation, the three divergent frequency ultrasonic of 20 kHz, 28 kHz and 35 kHz was applied and the triple frequency ultrasonic time was 20 s, 20 s and 20 s, respectively; ultrasonic power was 16.67 W/L, the initial temperature was 30° C. for 30 min; enzymatic hydrolysis was carried out by adding alkaline protease of 4560 U/g, enzymatic hydrolysis process was maintained at pH 9, temperature of 50° C., enzymolysis time of 30 min, then boiling of enzyme in water bath 10 min, 10000 g centrifugation for 10 min, supernatant was collected, and was diluted 2 times to measure ACE inhibitory activity.

After measurement, the inhibition rate of ACE in the hydrolysate of ultrasonic treatment was 73.80%, compared with the control (1), the antihypertensive activity increased by 13.62%. Compared with the control (4), the antihypertensive activity increased by 4.29% and the power density was reduced by 53.75 times. Compared with the control (5), the antihypertensive activity increased by 5.32% and the power density was reduced by 3 times.

### Implementation Example 2

Preparation of 3% (W/W) of the prion protein suspension of 6 L, under the condition of mechanical agitation, the three divergent frequency ultrasonic of 20 kHz, 28 kHz and 35 kHz was applied and the triple frequency ultrasonic time was 20 s, 20 s and 20 s, respectively; ultrasonic power is 16.67 W/L, the initial temperature of 30° C. of divergence type ultrasonic treatment for 30 min; enzymatic hydrolysis was carried out by adding alkaline protease of 4560 U/g, enzymatic hydrolysis process was maintained at pH 9, temperature of 50° C., enzymolysis time of 30 min, then boiling enzyme in water bath for 10 min, 10000 g centrifugation for 10 min, supernatant was collected and was diluted 3 times to measure ACE inhibitory activity.

After measurement, the inhibition rate of ACE in the hydrolysate of ultrasonic treatment was 77.73%, compared with the control 1-(2), the antihypertensive activity increased by 17.75%.

### Implementation Example 3

Preparation of 3% (W/W) of the prion protein suspension of 6 L, under the condition of mechanical agitation, the three divergent frequency ultrasonic of 20 kHz, 28 kHz and 35 kHz was applied and the triple frequency ultrasonic time was 20 s, 20 s and 20 s, respectively; ultrasonic power is 16.67 W/L, the initial temperature of 30° C. of divergence type ultrasonic treatment for 50 min; enzymatic hydrolysis was carried out by adding alkaline protease of 4560 U/g, enzymatic hydrolysis process was maintained at pH 9, temperature of 50° C., enzymolysis time of 30 min, then boiling enzyme in water bath for 10 min, 10000 g centrifugation for 10 min, supernatant was collected, and was diluted 3 times to measure ACE inhibitory activity.

After measurement, the inhibition rate of ACE in the hydrolysate of ultrasonic treatment was 76.76%, compared with the control 1-(3), the antihypertensive activity increased by 18.53%.

Plan Two:

The invention relates to a reverberation field: The reverberation field is the ultrasonic wave with a frequency of 20 kHz˜50 kHz, that has small absorption in liquid medium of aqueous solution and must be reflected in the inner wall of the vessel and the multiple reflection of the liquid surface in the sonochemical reactor. These frequencies are the same, but the random sound of the random phase changes in the space of a superposition of the sound field, the total sound intensity is equal to the sum of the sound intensity of each column, That is, there is no interference between them. According to the definition, when there are different interfaces, reflection will occur and the reverberation field will be formed. The invention designs three interfaces of solid-liquid, liquid-liquid and gas-liquid respectively to form ultrasonic reverberation field with different shapes, the solid-liquid interface is formed with non working ultrasonic plate and water body, the liquid-liquid interface is formed with vegetable oil and water body. The gas-liquid interface is formed by air and water body.

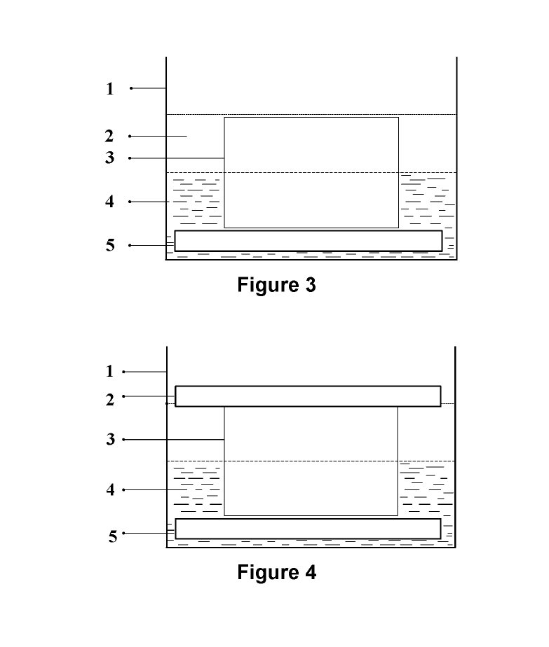

FIG. 2 is a liquid-liquid transmission medium ultrasonic reverberation field used in the invention. 1 is the reaction tank, 2 is oil phase (formed from vegetable oil), 4 is aqueous phase (formed by distilled water), Liquid-liquid transmission interface is formed on the contact surface between 2 and 4. 5 is the ultrasonic plate, which can issue 22±2 kHz, 28±2 kHz, 33±2 kHz, 40±2 kHz and 68±2 kHz frequency ultrasound. Ultrasonic mode is the sweep frequency ultrasound mode, power of 600 W. 3 is a sample bag, placed in the middle of 2 and 4, undergoing ultrasonic pretreatment in liquid-liquid ultrasonic reverberation field. FIG. 3 is a gas-liquid transmission medium ultrasonic reverberation field used in the invention. 1 is the reaction tank, 2 is gas (formed by air), 4 is aqueous phase (formed by distilled water), gas-liquid transmission interface is formed on the contact surface between 2 and 4. 5 is the ultrasonic plate, which can issue 22±2 kHz, 28±2 kHz, 33±2 kHz, 40±2 kHz and 68±2 kHz frequency ultrasound. Ultrasonic mode is the sweep frequency ultrasound mode, power of 600 W. 3 is a sample bag, placed in the middle of 2 and 4, undergoing ultrasonic pretreatment in gas-liquid ultrasonic reverberation field. FIG. 4 is a solid-liquid transmission medium ultrasonic reverberation field used in the invention. 1 is the reaction tank, 2 is the solid phase (formed by the non-working ultrasonic plate), 4 is aqueous phase (formed by distilled water), solid-liquid transmission interface is formed on the contact surface between 2 and 4. 5 is the ultrasonic plate, which can issue 22±2 kHz, 28±2 kHz, 33±2 kHz, 40±2 kHz and 68±2 kHz frequency ultrasound. Ultrasonic mode is the sweeping frequency ultrasound, power of 600 W. 3 is a sample bag, placed in the middle of 2 and 4, undergoing ultrasonic pretreatment in solid-liquid ultrasonic reverberation field.

Control Group 2:

Conventional enzymatic hydrolysis: preparation of corn protein suspension with concentration of 45 g/L was 180 mL volume, stirring for 30 min at a temperature of 18° C., addition of alkaline protease to carry out enzymatic hydrolysis, The reaction conditions were as follows: the enzyme dosage was 5616 U/g (E/S), enzymatic hydrolysis time of 60 min, enzymolysis temperature of 50° C., pH 9, use of 0.2 mol/L HCl to adjust pH to 7.0, enzyme inactivation in boiling water bath for 10 minutes, after cooling, centrifugation at 10000 g for 10 minutes, and then collecting the supernatant fraction, so far, the extract of protein antihypertensive peptide can be obtained. After enzymatic hydrolysis, use of 0.2 mol/L HCl to adjust pH to 7.0, enzyme inactivation in boiling water bath for 10 minutes, after cooling, centrifugation at 10000 g for 10 minutes, then collecting the supernatant and continuously varying to 800 ml and diluted for more than 5 times to determine the inhibitory activity of ACE. PH-stat was used to control pH and the DH value was measured during the experiment. Three replicates tests were done, after measurement, the inhibition rate of ACE of corn protein hydrolysate were 19.81% and DH was 7.52%.

### Implementation Example 4

Design of ultrasonic reverberation field of solid-liquid transmission medium, the solid-liquid interface is formed by the ultrasonic plate without work and water bodies. Preparation of corn protein suspension with concentration of 45 g/L was 180 mL volume, subjected to ultrasonic pretreatment, ultrasonic mode of operation is the sweeping mode (40±2) kHz, the processing conditions are as follows: the initial temperature is 18° C., the working time of the pulse ultrasonic is 10 s and the intermittent time is 3 s, the ultrasonic scanning period is 500 ms, the ultrasonic treatment time is 30 min, the ultrasonic power of the unit volume is 120 W/L. The hydrolysate that is treated by ultrasonic is hydrolyzed by alkaline protease, the reaction conditions are as follows: the substrate concentration is 4.5%, the amount of enzyme added is 5616 U/g (E/S), the enzymolysis time is 60 min, the hydrolysis temperature is 50° C., and the enzymatic hydrolysis is pH 9. After enzymatic hydrolysis, use of 0.2 mol/L HCl to adjust pH to 7.0, enzyme inactivation in boiling water bath for 10 minutes, after cooling, centrifugation at 10000 g for 10 minutes, then collecting the supernatant and continuously varying to 800 ml and diluted for more than 5 times to determine the inhibitory activity of ACE. The inhibition rate of ACE is 25.68% and DH is 8.81%. Compared to the controls, the inhibition rate of ACE is increased by 29.63%, and DH is increased by 15.8%.

### Implementation Example 5

Design of ultrasonic reverberation field of gas-liquid transmission medium, the gas-liquid interface is formed by air and water body. Preparation of corn protein suspension with concentration of 45 g/L was 180 mL volume, making ultrasonic pretreatment, ultrasonic process and enzymatic hydrolysis are the same as the implementation example (1). The inhibition rate of ACE is 23.96% and DH is 9.24%. Compared to the controls, the inhibition rate of ACE is increased by 20.94%, and DH is increased by 21.4%.

### Implementation Example 6

Design of ultrasonic reverberation field of liquid-liquid transmission medium, the liquid-liquid interface is formed by oil and water body. Preparation of corn protein suspension with concentration of 45 g/L was 180 mL, subjected to ultrasonic pretreatment, ultrasonic process and enzymatic hydrolysis are the same as the implementation example (1). The inhibition rate of ACE is 23.96% and DH is 9.24%. Compared to the controls, the inhibition rate of ACE decreased by 19.08%, DH decreased by 2.6%. It is easy to produce obvious emulsification in the ultrasonic reverberation field formed at the liquid-liquid interface, therefore, this kind of interface should be avoided in experiment and practical production, which has certain reference value for the design of instrument and equipment and industrial production.

### Implementation Example 7

Preparation of corn protein suspension of 180 mL volume with a concentration of 45 g/L. That is, in the solid-liquid scanning ultrasonic reverberation field ultrasonic time for 30 min. The ultrasonic frequency is 22±2 kHz, the initial temperature is 18° C., the working time of the ultrasonic pulse is 10 s, the intermittent time is 3 s, the sweeping period is 500 ms, the ultrasonic power of the unit volume is 120 W/L. Addition of alkaline protease to carry out enzymatic hydrolysis. Reaction conditions are the same as the control example. After enzymatic hydrolysis, use of 0.2 moL/L HCl to adjust pH to 7.0, enzyme inactivation in boiling water bath for 10 minutes, after cooling, centrifugation at 10000 g for 10 minutes, then collecting the supernatant and continuously varying to 800 mL and diluted for more than 5 times to determine the inhibitory activity of ACE. PH-stat was used to control pH and the DH value was measured during the experiment. Three replicates tests were done, after measurement, the inhibition rate of ACE of corn protein hydrolysate is 41.38%, and DH is 10.22%, respectively.

### Implementation Example 8

The experimental procedure is identical to the case 7, the difference is the sweeping frequency of 28±2 kHz. After measurement, the inhibition rate of ACE of hydrolysate of corn protein hydrolysate is 22.25% and DH is 10.15%.

### Implementation Example 9

The experimental procedure is identical to the case 7, the difference is the sweeping frequency of 33±2 kHz. After measurement, the inhibition rate of ACE of corn protein hydrolysate is 29.94% and DH is 9.91%.

### Implementation Example 10

The experimental procedure is identical to the case 7, the difference is the sweeping frequency of 40±2 kHz. After measurement, the inhibition rate of ACE of corn protein hydrolysate is 42.93% and DH is 10.35%.

### Implementation Example 11

The experimental procedure is identical to the case 7, the difference is the sweeping frequency of 68±2 kHz. After measurement, the inhibition rate of ACE of corn protein hydrolysate is 32.24% and DH is 9.87%.

Plan Three:

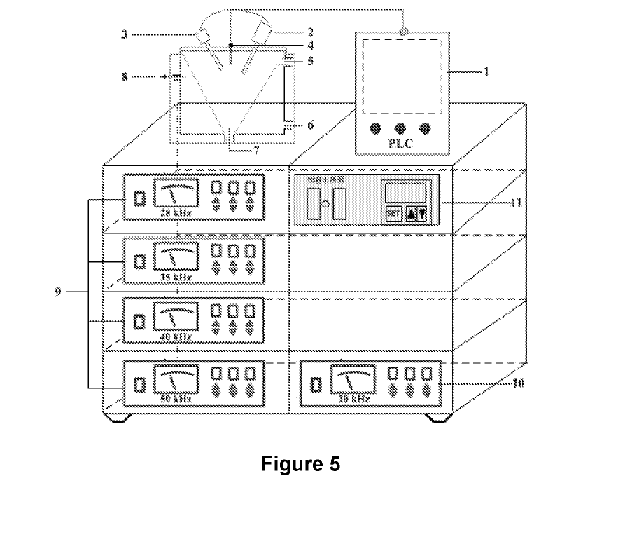

In FIG. 5, 1 is the multi-mode ultrasonic intelligent control panel, which can control the single frequency operation of the ultrasonic wave, the dual frequency sequential work and the dual frequency simultaneous work. 10 is the base frequency of 20 kHz ultrasonic generator. 9 are 28, 35, 40, 50 kHz ultrasonic generator, a single ultrasonic generator power of 100 W. 2 is the base frequency of 20 kHz ultrasonic probe, 3 is another combination probe. 7 is the material inlet, 5 is the liquid outlet. 6 is control warm water inlet, 8 is the control of warm water outlet. 11 is the temperature control system.

Control Group 3:

Conventional enzymatic hydrolysis: Preparation of 3% (W/W) of the prion protein suspension of 1 L by using a dilute solution of 0.003 mol/L, put into the 30° C. constant temperature water bath pot with rotor stirring for 15 min, addition of 4560 U/g (E/S) alkaline protease was used for enzymatic hydrolysis, during enzymatic hydrolysis process was maintained at pH 9, temperature of 50° C., enzymolysis time of 30 min, then boiling of enzyme in water bath for 10 min, 10000 g centrifugation for 10 min, supernatant was collected, and was diluted 3 times to measure ACE inhibitory activity. After measurement, the inhibition rate of ACE of gluten hydrolysate was 63.9%.

Control Group 4:

20 kHz single frequency ultrasound pretreatment assisted enzymatic hydrolysis. Conventional enzymatic hydrolysis: Preparation of 3% (W/W) of the prion protein suspension of 1 L by using a dilute solution of 0.003 mol/L, adopting 20 kHz single frequency probe type ultrasonic to treat wheat gluten protein suspension, the initial temperature is 30° C., the power is 200 W/L, the pulse time is 10 s, the intermittent time is 5 s, and the pretreatment time is 15 min. After the end of ultrasound, 4560 U/g (E/S) alkaline protease is used for enzymatic hydrolysis, during enzymatic hydrolysis process is maintained at pH 9, temperature of 50° C., enzymolysis time of 30 min, then boiling enzyme in water bath for 10 min, 10000 g centrifugation for 10 min, supernatant is collected, and is diluted 3 times to measure ACE inhibitory activity. After measurement, the inhibition rate of ACE gluten hydrolysate was 70.2%.

Control Group 5:

28 kHz single frequency ultrasound pretreatment assisted enzymatic hydrolysis. The experimental procedure is identical to the case 4, the difference is that the single frequency is 28 kHz. After measurement, the inhibition rate of ACE of gluten hydrolysate was 69.3%.

Control Group 6:

35 kHz single frequency ultrasound pretreatment assisted enzymatic hydrolysis. The experimental procedure is identical to the case 4, the difference is that the single frequency is 35 kHz. After measurement, the inhibition rate of ACE of hydrolysate of gluten hydrolysate was 71.5%.

Control Group 7:

40 kHz single frequency ultrasound pretreatment assisted enzymatic hydrolysis. The experimental procedure is identical to the case 4, the difference is that the single frequency is 40 kHz. After measurement, the inhibition rate of ACE of hydrolysate of gluten hydrolysate was 68.1%.

Control Group 8:

50 kHz single frequency ultrasound pretreatment assisted enzymatic hydrolysis. The experimental procedure is identical to the case 4, the difference is that the single frequency is 50 kHz. After measurement, the inhibition rate of ACE of gluten hydrolysate was 68.8%.

### Implementation Example 12

Preparation of 3% (W/W) of the prion protein suspension of 1 L by using a dilute solution of 0.003 mol/L, adopting the counter current sequential dual frequency ultrasound with a frequency of 20-28 kHz to pretreat the prion protein suspension, the pulsed time is 5 s and the working time is 5 s; the power is 200 W/L, the initial temperature is 30° C., and the pretreatment time is 15 min. After the end of ultrasound, 4560 U/g (E/S) alkaline protease is used for enzymatic hydrolysis, during enzymatic hydrolysis process is maintained at pH 9, temperature of 50° C., enzymolysis time of 30 min, then boiling enzyme in water bath for 10 min, 10000 g centrifugation for 10 min, supernatant was collected, then the extract of protein antihypertensive peptide is obtained, which is diluted 3 times to measure ACE inhibitory activity.

After measurement, the inhibition rate of ACE of gluten hydrolysate is 75.8%. Compared with control 1, the inhibition rate of ACE is increased by 18.6%. Compared with the control group 4 and 5, the inhibition rate was increased by 8% and by 9.4%, respectively.

### Implementation Example 13

The experimental procedure is identical to the case 12, the difference is that the frequency combination of the counter current type double frequency ultrasonic is 20-35 kHz. After measurement, the inhibition rate of ACE of gluten hydrolysate is 80.1%. Compared with control 1, the inhibition rate of ACE is increased by 25.4%. Compared with the control group 4 and 6, the inhibition rate was increased by 14.1% and by 12.0%, respectively.

### Implementation Example 14

The experimental procedure is identical to the case 12, the difference is that the frequency combination of the counter current type double frequency ultrasonic is 20-40 kHz. After measurement, the inhibition rate of ACE of gluten hydrolysate is 75.6%. Compared with control 1, the inhibition rate of ACE is increased by 18.3%. Compared with the control group 4 and 7, the inhibition rate was increased by 7.7% and by 11.0%, respectively.

### Implementation Example 15

The experimental procedure is identical to the case 12, the difference is that the frequency combination of the counter current type double frequency ultrasonic is 20-50 kHz. After measurement, the inhibition rate of ACE of gluten hydrolysate is 74.4%. Compared with control 1, the inhibition rate of ACE is increased by 16.4%. Compared with the control group 4 and 8, the inhibition rate is increased by 6.0% and by 8.1%, respectively.

### Implementation Example 16

Preparation of 3% (W/W) of the prion protein suspension of 1 L by using a dilute solution of 0.003 mol/L, adopting the counter current sequential dual frequency ultrasound with a frequency of 20-28 kHz to pretreat the prion protein suspension, the pulse duration was 10 s and the intermittent time was 5 s. Each ultrasonic frequency has power of 100 W/L and the total ultrasonic power of dual frequencies is 200 W/L. The initial temperature is 30° C., and the pretreatment time is 15 min. After the end of ultrasound, 4560 U/g (E/S) alkaline protease is used for enzymatic hydrolysis, during enzymatic hydrolysis, process is maintained at pH 9, temperature of 50° C., enzymolysis time of 30 min, then boiling enzyme in water bath for 10 min, 10000 g centrifugation for 10 min, supernatant was collected, the extract of protein antihypertensive peptide is obtained, and is diluted 3 times to measure ACE inhibitory activity.

After measurement, the inhibition rate of ACE of gluten hydrolysate is 78.8%. Compared with the control 1, the antihypertensive activity increased by 23.3%, compared with the control group 4 and 8, the inhibition rate was increased by 12.3% and by 13.7%, respectively.

### Implementation Example 17

The experimental procedure is identical to the case 16, the difference is that the frequency combination of the counter current type double frequency ultrasonic is 20-35 kHz. After measurement, the inhibition rate of ACE of hydrolysate of gluten hydrolysate is 76.6%. Compared with control 1, the antihypertensive activity increased by 19.9%, compared with the control group 4 and 6, the inhibition rate was increased by 9.1% and by 7.1%, respectively.

### Implementation Example 18

The experimental procedure is identical to the case 16, the difference is that the frequency combination of the counter current type double frequency ultrasonic is 20-40 kHz. After measurement, the inhibition rate of ACE of gluten hydrolysate is 75.1%. Compared with control 1, the antihypertensive activity increased by 17.5%, compared with the control group 4 and 7, the inhibition rate of was increased by 7.0% and by 10.2%, respectively.

### Implementation Example 19

The experimental procedure is identical to the case 12, the difference is that the frequency combination of the counter current type double frequency ultrasonic is 20-50 kHz. After measurement, the inhibition rate of ACE of gluten hydrolysate is 74.5%. Compared with control 1, the antihypertensive activity increased by 16.6%, compared with the control group 4 and 8, the inhibition rate was increased by 6.1% and by 8.3%, respectively.

1. A method for preparing functional polypeptide by multi-mode ultrasonic-assisted enzymolysis, and method for preparing blood pressure reducing peptide of protein of wheat gluten by sequential ultrasonic intensification, comprising the following steps: (1) the gluten is formulated as a suspension with a mass concentration of 3%, and pretreated by the order of triple frequency ultrasound. (2) under the conditions of pH 9.0, temperature of 50° C., substrate concentration of 3%, alkaline protease enzyme dosage 4560 U/g, enzymatic hydrolysis for 30 minutes, use of 0.2 mol/L HCl to adjust pH to 7.0, enzyme inactivation in boiling water bath for 10 minutes, after cooling, centrifuge at 10000 g for 10 minutes, and then collecting the supernatant fraction, so far, the extract of protein antihypertensive peptide is obtained.

2. The method for the preparation of functional polypeptides according to the multi-mode ultrasonic assisted enzymatic hydrolysis described in claim 1, wherein the order of triple ultrasonic frequency is 20 kHz, 28 kHz and 35 kHz; triple ultrasonic time is 20 s, 20 s, 20 s, and the ultrasonic power per unit volume is 16.67 W/L, pretreatment time 10-16 minutes.

3. A method for preparing functional polypeptide by multi-mode ultrasonic wave strengthening enzymolysis, and method for preparing corn protein antihypertensive peptide by means of scanning frequency reverberation field ultrasonic strengthening enzymolysis, comprising the following steps: (1) suspension with mass concentration of 4.5%, which is made from corn protein, and pretreated by the reverberation field ultrasonic; (2) under the conditions of pH 9.0, temperature of 50° C., substrate concentration of 4.5%, alkaline protease enzyme dosage 5616 U/g, enzymatic hydrolysis for 60 minutes, use of 0.2 mol/L HCl to adjust pH to 7.0, enzyme inactivation in boiling water bath for 10 minutes, after cooling, centrifuge at 10000 g for 10 minutes, and then collecting the supernatant fraction, so far, the extract of protein antihypertensive peptide is obtained.

4. The method for the preparation of functional polypeptides according to the multi-mode ultrasonic enhanced enzymatic hydrolysis described in claim 3, wherein a sweep frequency is 22±2 kHz, 28±2 kHz, 33±2 kHz, 40±2 kHz or 68±2 kHz.

5. The method for the preparation of functional polypeptides according to the multi-mode ultrasonic enhanced enzymatic hydrolysis described in claim 3, wherein a sweep frequency form reverberation field with different reflection media through the three interfaces of solid liquid, liquid liquid and gas liquid, the solid liquid interface is formed by metal plates and water bodies, the liquid liquid interface is formed by vegetable oil and water, the gas-liquid interface is formed by air and water.

6. The method for the preparation of functional polypeptides according to the multi-mode ultrasonic enhanced enzymatic hydrolysis described in claim 3, wherein ultrasonic reverberation prefer solid liquid medium; sweep frequency of ultrasonic reverberation field is (40±2) kHz.

7. The method for the preparation of functional polypeptides according to the multi-mode ultrasonic enhanced enzymatic hydrolysis described in claim 3, wherein ultrasonic pretreatment conditions include: the working time of pulsed ultrasound is 10 s and intermittent time of 3 s, ultrasonic sweep period 500 ms, unit volume ultrasonic power 120 W/L, initial temperature of 18 C, pretreatment time of 30 minutes.

8. A method for preparing functional polypeptide by multi-mode ultrasonic intensified enzymolysis, and method for preparing blood pressure reducing peptide of protein of wheat gluten by counter current dual frequency ultrasound, comprising the following steps: (1) Making wheat gluten into gluten protein suspension with mass concentration of 3% with dilute alkali solution of 0.003 mol/L, and pretreated by counter current dual frequency ultrasound; (2) under the conditions of pH 9.0, temperature of 50° C., alkaline protease (Alcalase) enzyme dosage 4560 U/g, substrate concentration of 10 g/L, enzymatic hydrolysis for 30 minutes, use of 0.2 mol/L HCl to adjust pH to 7.0, enzyme inactivation in boiling water bath for 10 minutes, after cooling, centrifuge at 10000 g for 10 minutes, and then collecting the supernatant fraction, so far, the extract of protein antihypertensive peptide is obtained.

9. The method for the preparation of functional polypeptides according to the multi-mode ultrasonic enhanced enzymatic hydrolysis described in claim 8, wherein the mode of operation of the ultrasonic is the order of dual frequency and synchronous dual frequency, frequency combination is 20-28, 20-35, 20-40 and 20-50 kHz, the frequency of the sequential operation is 20-35 kHz, and the working time is 5 s and pulsed time is 5 s, the frequency of the synchronous operation is 20-28 kHz, the pulse time is 10 s, the intermittent time is 5 s.

10. The method for the preparation of functional polypeptides according to the multi-mode ultrasonic enhanced enzymatic hydrolysis described in claim 8, wherein ultrasonic pretreatment conditions include: the ultrasonic power per unit volume is 200 W/L, the initial temperature is 30° C., and the pretreatment time is 15 min.
